(19) United States
(12) Patent Application Publication
(10) Pub. No.: US 2004/0028723 A1
(43) Pub. Date: Feb. 12, 2004

(54) Transdermal therapeutic system for treating restless-legs-syndrome

(76) Inventors: Reinhard Horowski, Berlin (DE); Johannes Tack, Berlin (DE); Adalbert Engfer, Berlin (DE)

Correspondence Address: Wood Phillips Katz Clark & Mortimer, 500 West Madison Street, Suite 3800, Chicago, IL, 60661, US

(21) Appl. No.: 10/362,183

(22) PCT Filed: Aug. 24, 2001
(86) PCT No.: PCT/EP01/09823

(30) Foreign Application Priority Data
Aug. 24, 2000 (DE) 10043321.9

(51) Int. Cl. A61K 31/48; A61K 9/70

(52) U.S. Cl. 424/449; 514/288

(57) ABSTRACT

The invention relates to the use of a transdermal therapeutic system (TTS) comprising a medicinal layer, which contains at least one matrix comprising an active ingredient and/or an active ingredient reservoir and a diffusion barrier situated on the skin side of the active ingredient reservoir and permeable to active ingredients, in addition to, an ergoline-derivative or physiologically compatible salt with an acid thereof, as an active ingredient, for producing a means for treating the restless-legs-syndrome.

This invention relates to a transdermal therapeutic system (TTS) comprising a pharmaceutical layer containing at least one matrix having an active ingredient and/or an active ingredient reservoir; a diffusion barrier that is permeable to said active ingredient and arranged on the skin side of the active ingredient reservoir; and an ergoline derivative or salt thereof as an active ingredient to produce an agent for treating restless legs syndrome.

Restless legs syndrome (RLS) is a neurological disease that can occur at all ages but is more frequent in older people; its main symptoms are cramps and pain in the legs due to dysesthesias and paresthesias that trigger an urge to move. As these symptoms mostly occur in periods of reduced activity such as when sitting or resting, the urge to move results in restlessness during the day and sleep disturbances at night. This considerably impairs the quality of life of those affected.

It is known that treating restless legs syndrome with single oral administrations of dopaminergic drugs such as lisuride in the evening reduces the symptoms and has a positive influence on the patients quality of life. Unlike the treatment of Parkinson's disease where dopaminergic pharmaceuticals and combinations thereof are administered throughout the day, one-time peroral intake of these drugs for the treatment of restless legs syndrome impairs the building of a tolerance against acute dopaminergic side effects (due to the initial flux rate); this means that the known side effects such as orthostasis, hypotonia, dizziness, nausea, and vomiting may occur with each effective dose. Unpredictable and uncontrollable sleep attacks that have recently been reported more frequently may also occur. Furthermore, agent concentration in the plasma is not constant but subject to great variation, not only for kinetic reasons but also depending on the conditions of drug intake (type and time of food intake, etc.). This is why there is a risk of temporary overdosing, which may result in REM suppression and the resulting problems and sleep disturbances.

In addition, peroral dopaminergic therapies often lead to rebound problems on the following day and to so-called augmentations, i.e. hypertonus, restlessness and an urge to move.

It is the technological problem of this invention to provide an agent for the treatment of restless legs syndrome that is free of side effects or at least shows considerably reduced side effects as compared to oral administrations, that has a slow initial flux rate and can be controlled well in terms of quantity administered and effective time.

A transdermal therapeutic system according to the invention described below can ensure an individually desired and controlled effective time (if required, by removing the patch). Bioavailability is increased by the TTS as compared to peroral administration, which typically reduces the overall dose required to achieve the therapeutically desirable effect. The α-adrenolytic effect of lisuride and its derivatives has another benefit with this form of application in that it also noticeably diminishes urinary urgency at nighttime and other bladder dysfunctions that are rather common in Parkinson patients (such as prostatic hyperplasia), which adds to the success of the therapy.

The invention relates to the use of a transdermal therapeutic system (TTS) comprising a pharmaceutical layer containing at least one matrix having an active ingredient, and/or an active ingredient reservoir; a diffusion barrier which is permeable to active ingredients and which is arranged on the skin side of the active ingredient reservoir; and an ergoline derivative according to formula I or physiologically compatible salt thereof with an acid,

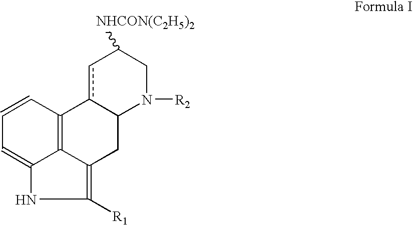

wherein [symbol] is a single or double bond wherein R1 is an H atom or a halogen atom, particularly a bromine atom, and wherein R2 is C1-C4 alkyl, particularly methyl, as an agent for treating restless leg syndrome.

A special benefit this invention offers is that—other than with the common one-time oral intake per day —a continuous active ingredient flux is established so that plasma concentrations can be set as defined and variations can be controlled. This mainly prevents the side effects typically observed with one-time oral administration such as fatigue, dizziness, vomiting, constipation, etc. It was found that these side effects can be prevented when the level of active ingredient in the plasma is not subject to any major and rapid variation, an automatic occurrence with oral administration, but is set slowly and continuously. In addition, the problems encountered with oral administration such as greatly varying absorption rates and a not too well-defined time of maximum concentration in the plasma depending on the type and time of food intake are virtually eliminated by this invention. Most of all, it prevents overdosing (and thus REM suppression and other disruptions of the sleep pattern). Furthermore, administration can easily be canceled by just removing the TTS. Unlike discontinuing an orally administered active agent, decomposition in the plasma is fast and controlled, which also prevents a hangover, rebound, or augmentation effect. Finally it is easy to administer exact individual doses by selecting the flux F and/or the active surface area. It is preferred to select F and active area so that a dose in the range from 10 μg to 2 mg of active ingredient (for example, lisuride), most preferred 50 to 200 μg, is built up per day.

It is preferred that the matrix and/or diffusion barrier are selected so that the transdermal flux F through human skin measured as described in Example 1 is in the range from 0.1 to 2.0 μg/cm²/h.

The list of ergoline derivatives that can be used includes the following: Bromolisuride (3-(2-bromo-9,10-didehydro-6-methyl8α-ergolinyl)-1,1-diethyl urea), terguride (3-(6-methyl-8α-ergolinyl)-1,1-diethyl urea) and proterguride (3-(6-propyl-8α-ergolinyl)-1,1-diethyl urea). However it is preferred when the ergoline derivative is lisuride (3-(9,10-didehydro-6-methyl-8α-ergolinyl)-1,1-diethyl urea) or its physiologically compatible salt with an acid. The production of lisuride and other suitable ergolines according to the invention is described, inter alia, in U.S. Pat. No. 3,953,454, EP 056 358 and U.S. Pat. No. 4,379,790. Suitable salts of the ergoline derivative include sulfates, phosphates, maleates, citrates and succinates, especially hydrogen maleate.

The term “TTS” mostly denotes percutaneously acting but also transmucosal systems. A TTS typically has a sheet-like structure and is attached to an area of the skin. The system can optionally be attached to the skin by an additional skin-side adhesive that is permeable to the active ingredient. Alternatively, the matrix and/or diffusion barrier can itself have adhesive properties. And finally a non-adhesive TTS can be attached to the skin using other auxiliary means such as adhesive tapes or bandages. The matrix is a material in which the active ingredient is immobilized. An active agent in an active ingredient reservoir however is not necessarily immobilized, which is why the active ingredient reservoir must be enclosed. The diffusion barrier forms the skin-side portion of this shell. It goes without saying that all other parts of the shell should be as impermeable as possible, including diffusion paths, to the active ingredient. Immobilized means in this context that any uncontrolled active ingredient flow is prevented. However diffusion of an active agent in a matrix and/or through a diffusion barrier is not only possible but intended. The diffusion coefficients eventually determine the active ingredient flux from the TTS into a patient's skin. The dose released into a patient's skin is in first approximation a linear function of the active area of the TTS. The active area is the contact area of those TTS portions that allow active ingredient diffusion. TTSs can be used in human and veterinary medicine.

A TTS of the design mentioned above is known in principle from publication WO 92/20339. It specifically describes the effect of propylene glycol lauric acid on the flux, i.e. a considerable increase in flux. However the values specified therein relate to solutions applied to skin samples and not to the actual TTS. No specification is given regarding flux from a TTS. The flux values reached with a TTS are considerably lower than the values from applying a solution.

A TTS containing lisuride is further known from publication WO 91/00746. The flux values for human skin samples specified therein cannot be directly transferred to any achievable in-vivo values.

TTSs of the design described above are used for various indications including Parkinson's disease. When treating Parkinson's disease, the highest possible doses are desirable. A transdermal therapeutic system also improves compliance, which is of critical importance for any combinatory treatment of this disease as patients tend to be older and have multiple diseases. Improved control and the chance to reach circadian profiles (e.g. by low stimulation as constantly as possible at night or during a break) are particularly important and have not yet been achieved (e.g. to prevent psychoses and improve sleep quality). The ergoline derivatives lisuride, terguride, and bromerguride have a partially dopamine-agonistic or partially antagonistic effect that contributes to preventing the development of psychoses and can improve existing psychoses and similar problems.

The TTS can be designed as follows. A covering layer can be arranged on the side of the matrix and/or active ingredient reservoir facing away from the skin. It may be formed by films of polyethylene or polyester. It is typically 10 to 100 microns in thickness. The covering layer may be pigmented and/or metal plated to ensure sufficient protection from light. Metal plating involves applying a very thin layer (typically less than 1 micron, mostly in the 10-100 nm range) of a metal such as aluminum to the covering layer. Pigments can be all pigments commonly used for coating including effect pigments as long as these are physiologically harmless. A detachable liner such as a siliconized or fluoropolymer-coated protective film can be provided on the application side.

The matrix and/or diffusion barrier may comprise as their main matrix component a substance selected from the group consisting of “polyacrylate, polyurethane, cellulose ether, silicone, polyvinyl compounds, silicate and mixtures of these substances as well as copolymers of these polymeric compounds,” preferably polyacrylate. A main matrix component makes up at least 50 percent by weight, e.g. at least 80-90 percent by weight of the matrix (matrix to be understood as the finished layer, i.e. main matrix component(s) with adjuvant(s) and active ingredient(s)). The desired flux is set by selecting the substance depending on the diffusion coefficient of the active ingredient and, if required, by selecting the layer thickness of the matrix in orthogonal direction to the skin surface. Matrix thickness is typically in the range from 10 to 500 microns.

A preferred polyacrylate adhesive as main matrix component is commercially available under the brand name GELVA® multipolymer solution 7881, provided by Monsanto Deutschland GmbH, Düsseldorf. We expressly refer to the product sold under this name and its datasheet in the version of Apr. 23, 1996. Another suitable product is Eudragit® E100 provided by Röhm, Germany.

The polyacrylate adhesives listed above provide an advantageous non-trivial combination of properties, namely optimum flux, good adhesive power, good skin compatibility, and durability.

The diffusion barrier can alternatively comprise as its main barrier component a polymer selected from the group consisting of “cellulose ester, cellulose ether, silicone, polyolefin and mixtures as well as copolymers of these substances.” What has been said about the term of the main matrix component above analogously applies to the term of the main barrier component.

The diffusion barrier can be a film with a thickness from 10 to 300 microns; the actual film thickness is selected (in conjunction with the diffusion coefficient of the active ingredient in the polymer) according to the desired flux.

The matrix and/or active ingredient reservoir and/or diffusion barrier may contain the common adjuvants used in TTSs. It is preferred to use a penetration-enhancing agent that is preferably selected from the group consisting of “C1-C8 aliphatic, cycloaliphatic and aromatic alcohols, saturated and unsaturated C8-18 fatty alcohols, saturated and unsaturated C8-18 fatty acids, hydrocarbons and hydrocarbon mixtures, fatty acid esters from C3-19 fatty acids and C1-6 alkyl monools, dicarboxylic acid dieesters from C4-8 dicarboxylic acids and C1-6 alkyl monools, and mixtures of these substances. Penetration-enhancing agents improve the flux of the active ingredient through the skin to which the TTS is attached. Examples of the substances listed above are: 1,2-propane diol, menthol, dexpanthenol, benzyl alcohol, lauryl alcohol, isocetyl alcohol, cetyl alcohol, mineral oil, lauric acid, isopalmitic acid, isostearic acid, oleic acid; methyl ester, ethyl ester, 2-hydroxyethyl ester, glycerol ester, propyl ester, isopropyl ester, butyl ester, sec. butyl ester or isobutyl ester of lauric acid, myristic acid, stearic acid, or palmitic acid. Use of dimethyl isosorbide, isopropyl myristate and lauryl alcohol is preferred, use of lauryl alcohol is most preferred. Other adjuvants are, for example, crystallization inhibitors. Suitable crystallization inhibitors are highly dispersed silicon dioxide or macromolecular substances such as polyvinyl pyrrolidone, polyvinyl alcohols, dextrines, dextranes, sterines, bile acids and, in particular, vinyl pyrrolidone vinylacetate copolymers such as Kollidon® VA 64.

It goes without saying that the penetration-enhancing agent has to be able to diffuse to a sufficient extent through the matrix or diffusion barrier. If a matrix and lauryl alcohol as an adjuvant are used, it is preferred that the lauryl alcohol makes up 10 to 30 percent by weight, most preferred 15 to 20 percent by weight, of the matrix.

The adjuvants can basically make up from 0 to 50 percent by weight of the matrix. The active ingredient can make up 0.2 to 20 percent by weight, preferably 1 to 10 percent by weight, of the matrix. The sum total of main matrix component, adjuvants and active ingredients is always 100 percent by weight.

The active ingredient dose in a human body carrying a TTS is dependent on the diffusion-related properties of the TTS mentioned above and also on its active surface area on the skin. Active surface area means the area over which the matrix or diffusion barrier comes to rest on the skin. Variation in accordance with the desired dosage will preferably be in a range from 1 to 100 cm².

Within the scope of this invention, a physician can easily set up personalized dose variations for a flux adjusted to the given indication by selecting a suitable patch size. Thus the treatment can easily be adjusted to different body weights, age groups, etc. It is particularly feasible to equip a TTS comprising a (rather large) standard area with subdivision markers for partial doses so that a user can just remove the protective film from a partial area corresponding to the specified dose. The respective subsections can easily be printed on the covering layer.

Another application is the use of a TTS according to the invention to produce an agent for the treatment or prevention of the premenstrual syndrome or its symptoms, wherein F preferably is in the range from 0.1 to 0.5 μg/cm²/h, another one to produce an agent that inhibits lactation, wherein F preferably is in the range from 0.1 to 0.5 μg/cm²/h.

The invention will be explained in more detail below based on various examples.

### EXAMPLE 1

### Flux Measurement

A FRANZ flow-through diffusion cell is used for flux measurement. The measured area is 2 cm². 4 cm² of ventral and dorsal skin of a male hairless mouse (MF1 hr/hr Ola/Hsd, provided by Harlan Olac, UK) are used as our skin sample after carefully removing any subcutaneous fatty tissue. A 2 cm² TTS is applied to the skin sample. The acceptor medium is placed on the opposite side. It is diluted HHBSS (Hepes Hanks Balanced Salt Solution) containing 5.96 g/l of Hepes, 0.35 g/l of NaHCO₃ and 0.1 ml/l 10× of HBSS (provided by Gibco, Eggenstein, DE). Furthermore, 1000 I.U./ml of penicillin (benzylpenicillin potassium salt, provided by Fluka, Neu-Ulm, DE) are used.

The flux is measured as described below. First, the TTS to be measured is applied to the skin. The skin is mounted in the diffusion cell immediately thereafter. Samples of the acceptor medium are taken at 2-hour intervals between t=0 hrs and t=6 hrs and at 8-hour intervals between t=6 hrs and t=54 hrs. 1 ml of acceptor medium per hour is pumped through the diffusion cell using a peristaltic pump. The temperature of the acceptor medium is controlled using a circulating water bath which keeps the skin at a temperature of 31° C. with an accuracy of 1° C.

The active ingredient concentration in the acceptor medium is determined in accordance with the following specifications using a radioimmunoassay.

Calibration curves: These are constructed using two different methanol solutions of non-radioactive lisuride hydrogen maleate salt, each containing 1 mg/ml. These solutions are individually diluted with BSA buffer (0.041 M of Na₂HPO₂*2H₂O, 0.026 M of KH₂PO4, 0.154 M of NaCl, 0.015 M of NaN₃, 0.1% (w/v) of BSA, pH 7, supplemented with 0.05% (w/v) of ascorbic acid) to obtain lisuride-free base concentrations in the range from 1000 - 3.9 pg/0.1 ml. In addition, a sample without active ingredient (0 pg) is used. The calibration samples are analyzed three times. The lisuride concentrations are calculated using the pharmacokinetic PC program RIO 2.5 (other common software may also be used).

Sample preparation: The acceptor medium is diluted with BSA buffer prior to the analysis to set the concentrations to an analyzable range of the calibration curve. 100 μl of diluted sample are directly subjected to radioimmunological analysis.

Antiserum: The antiserum (rabbit) is obtained by immunizing with lisuride-1-succinyl-BSA, an immunogen. The antiserum in the assay is diluted 1:12500.

Tracer: ³H-lisuride hydrogen maleate with a specific activity of 4.3 GBq/mg is used.

Incubation: 0.1 ml of BSA buffer with active ingredient, 0.1 ml of tracer solution (ca. 5000 cpm/0.1 ml of BSA buffer) and 0.1 ml of diluted antiserum (1:12500) are added to 0.7 ml of BSA buffer and incubated for 18 hours at 4° C.

Separation: antibody-bound lisuride is separated from free lisuride by adding 0.2 ml of charcoal suspension (1.25%(w/v) and 0.125% (w/v) of dextrane in BSA buffer) and incubation for 30 minutes at 0° C. The charcoal is sedimented by centrifuging for 15 minutes at 3000 g. The supernatant liquid (containing antibody-bound active ingredient) is decanted and subjected to radiometric analysis.

Radiometric analysis: 4 ml of Atomlight (NEN) scintillation cocktail are added to the supernatant. The count is carried out using a WALLAC 1409 or 1410 β-scintillation counter without quench control.

Analysis: The percutaneous skin flux is calculated as follows:

F=(C * R)/(A*T),

where F is the percutaneous flux [ng/cm²/h], C the active ingredient concentration in the acceptor medium [ng/ml], R the acceptor medium flow [1ml/h], A the measured area [2cm²] and T the sample-taking interval [h].

The maximum transdermal active ingredient flux is obtained directly from the data. Mean percutaneous flux values are determined during days 1 and 2 of the experiment based on the cumulative absorbed dose in time intervals t=0-22 and t=22-54.

Specifications for the production of TTS

### EXAMPLE 2

### TTS A

15 mg of Kollidon VA 64 (crystallization inhibitor) are dissolved in 15 mg of isopropanol. Then 5 mg of lisuride are sprinkled in. 80 mg of polyacrylate adhesive (Gelva 7881) are placed in a beaker, and the above suspension is added while rerinsing with 30 mg of isopropanol. The crystal-free wet mix obtained is thoroughly intermixed and spread on a siliconized liner using a 500 micron blade. The product is dried at 60° C. for 20 minutes, and finally a covering layer is laminated onto it.

Flux measurements as described in Example 1 showed an F value of 0.43 on day 1, 0.44 on day 2, and a maximum F value of 0.85 (each in μg/cm²/h).

### EXAMPLE 3

### TTS B

12.5 mg of dimethyl isosorbide are suspended with 2 mg of lisuride in 15 mg of isopropanol. 80 mg of polyacrylate adhesive (Gelva 7881) are placed in a beaker, and the above suspension is added while rerinsing with 30 mg of isopropanol. The crystal-free wet mix obtained is thoroughly intermixed and spread on a siliconized liner using a 500 micron blade. The product is dried at 60° C. for 20 minutes, and finally a covering layer is laminated onto it.

Flux measurements as described in Example 1 showed an F value of 0.23 on day 1, 0.28 on day 2, and a maximum F value of 0.50 (each in μg/cm²/h).

### EXAMPLE 4

### TTS C

27.2 mg of Kollidon VA 64 (crystallization inhibitor) and 16.3 mg of lauryl alcohol are dissolved at 60° C. Then 2 mg of lisuride are dissolved in this solution at 60° C. 39.38 mg of Eudragit E100, 13.41 mg of Citroflex 4A and 1.71 mg of succinic acid are molten at 150-200° C. The lisuride solution is added after the batch has cooled down to 80° C. The product is spread at 80° C. on a siliconized liner using a 500 micron blade. Then the product is cooled down to 20° C.; optionally, a covering layer may be laminated onto it.

Flux measurements as described in Example 1 showed an F value of 0.90 on day 1, 1.76 on day 2, and a maximum F value of 2.53 (each in μg/cm²/h).

1) Use of a transdermal therapeutic system (TTS) comprising a pharmaceutical layer containing at least one matrix having an active ingredient, and/or an active ingredient reservoir; a diffusion barrier which is permeable to active ingredients and which is arranged on the skin side of the active ingredient reservoir; and an ergoline derivative according to formula I or physiologically compatible salt thereof with an acid, 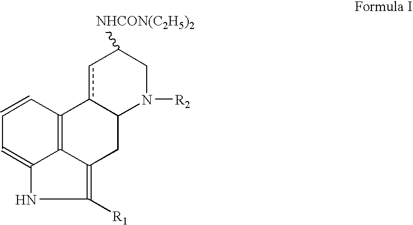
wherein [symbol] is a single or double bond wherein R1 is an H atom or a halogen atom, particularly a bromine atom, and wherein R2 is C1-C4 alkyl, particularly methyl, as an agent for treating restless leg syndrome.

2) The use according to claim 1 wherein the matrix and/or diffusion barrier are selected so that the transdermal flux F through human skin measured as described in Example 1 is in the range from 0.1 to 5.0 μg/cm²/h.

3) The use according to claims 1 or 2 wherein the ergoline derivative is lisuride or a salt thereof with a physiologically compatible acid.

4) The use according to any one of claims 1 through 3 wherein a covering layer is provided on the side of the matrix and/or active ingredient reservoir that faces away from the skin.

5) The use according to any one of claims 1 through 4 wherein the matrix and/or diffusion barrier comprise as their main matrix component a substance selected from the group consisting of “polyacrylate, polyurethane, cellulose ether, silicone, polyvinyl compounds, silicate and mixtures of these substances as well as copolymers of these polymeric compounds.”

6) The use according to any one of claims 1 through 5 wherein the diffusion barrier comprises as its main barrier component a synthetic polymer selected from the group consisting of “cellulose ester, cellulose ether, silicone, polyolefin and mixtures as well as copolymers of these substances.”

7) The use according to any one of claims 1 through 6 wherein the matrix and/or the active ingredient reservoir and/or the diffusion barrier contain a penetration-enhancing agent that is preferably selected from the group consisting of “C1-C8 aliphatic, cycloaliphatic and aromatic alcohols, saturated and unsaturated C8-18 fatty alcohols, saturated and unsaturated C8-18 fatty acids, hydrocarbons and hydrocarbon mixtures, fatty acid esters from C3-19 fatty acids and C1-6 alkyl monools, dicarboxylic acid diesters from C4-8 dicarboxylic acids and C1-6 alkyl monools, and mixtures of these substances.”

8) Use of a TTS according to any one of claims 1 through 7 to produce an agent for the treatment or prevention of premenstrual syndrome or its symptoms wherein the preferred F value is in the range from 0.1 to 0.5 μg/cm²/h.

9) Use of a TTS according to any one of claims 1 through 7 to produce an agent for lactation inhibition wherein the preferred F value is in the range from 0.1 to 0.5 μg/cm²/h.
